(19) United States
(12) Patent Application Publication
(10) Pub. No.: US 2023/0191038 A1
(43) Pub. Date: Jun. 22, 2023

(54) A DIAL GRIP FOR AN INJECTION DEVICE

(71) Applicant: Sanofi, Paris (FR)

(72) Inventor: Stefan Alt, Frankfurt am Main (DE)

(21) Appl. No.: 17/923,633

(22) PCT Filed: May 17, 2021
(86) PCT No.: PCT/EP2021/062914

§ 371 Date: Nov. 7, 2022

(30) Foreign Application Priority Data
May 18, 2020 (EP) 20315242.6

(51) Int. Cl. A61M 5/315

(52) U.S. Cl. CPC A61M 5/3155; A61M 2205/3584

(57) ABSTRACT

A dial grip of an injection device comprises a grip base, an handling part provided on one side of the grip base, and clutch being provided on the other side of the grip base for releasable engagement with a dose selecting part of a dosage selection and expelling mechanism of the injection device to couple the grip base to the dosage selection and expelling mechanism for selecting and expelling a dosage with the dial grip.

### CROSS REFERENCE TO RELATED APPLICATIONS

The present application is the national stage entry of International Patent Application No. PCT/EP2021/062914, filed on May 17, 2021, and claims priority to Application No. EP 20315242.6, filed on May 18, 2020, the disclosures of which are incorporated herein by reference.

### TECHNICAL FIELD

The present disclosure relates to a dial grip for an injection device.

### BACKGROUND

A variety of diseases exists that require regular treatment by delivery, particularly injection of a drug or medicament. Such injection can be performed by using injection devices, which are applied either by medical personnel or by patients themselves.

Injection devices may be disposable or reusable, such as the disposable Solostar™ injection pen and the reusable Allstar™ injection pen, both from Sanofi. The mechanical construction of the Allstar™ injection pen is described in detail in the international patent application WO2014/033195A1. An injection pen such as the Solostar™ and Allstar™ injection pens comprises a distal and a proximal end, wherein in the context of this disclosure the term proximal refers to the direction pointing towards the patient during an injection while the term distal refers to the opposite direction pointing away from the patient. A button or knob at the distal end of the Solostar™ and Allstar™ injection devices serves as a dial grip for selecting a dosage to be injected with the devices.

### SUMMARY

This disclosure describes different concepts of a dial grip for an injection device, particularly for a needle-based injection system for self-administration of drugs by patients. The dial grip may be provided for releasable attachment to an injection device and may be according to one embodiment used as dummy dial grip for example for shipping injection devices. The dial grip may be provided for dose selection with the injection device. The injection device may be a pen-shaped device having a distal and a proximal end, and the dial grip may form the distal end of the injection pen, which may be turned by a patient to select or set a custom dose. In some embodiments, the dial grip may be pushed inwardly by applying an axial force to the rear end of the dial grip by the patient to inject the selected or set dose. In other embodiments, a separate button may be provided that may be pushed inwardly by applying an axial force to the rear end of the button by the patient to inject the selected or set dose. The dial grip may be particularly provided for releasable attachment to the drug injection device so that it can be replaced with another dial grip. The dial grip may be attached to a reusable injection device such as the Allstar™ pen, which may comprise an exchangeable cartridge for a drug or medicament, or to a disposable injection device comprising a fixed amount of a drug or medicament within the device, such as the Solostar™ pen. Injection devices may be for example shipped with dummy dial grips, which can be passive dial grips, i.e., grips without any special functionality. A dummy dial grip may be understood herein as a dial grip, which may provide no functionality at all or at least no further functionality in addition to a basic functionality such as for example dosage selecting. It may particularly serve as a kind of securing device for shipping injection devices. A dummy dial grip may be releasably attached to an injection device so that it can be replaced with another dial grip. A dummy dial grip may be extended with a grip top or replaced by a replacement dial grip, the grip top or the replacement dial grip comprising special functionality such as for example dose recording and connectivity features or other utility features. Such other features for a more comfortable handling of the dial grip may be attached for example by a patient to the dummy dial grip in order extend the basic functionality of the dummy dial grip.

In one aspect the present disclosure provides a dial grip for an injection device, the dial grip comprising a grip base, an handling part provided on one side of the grip base, and a clutch being provided on the other side of the grip base for engagement with a dose selecting part of a dosage selection and expelling mechanism of the injection device to couple the grip base to the dosage selection and expelling mechanism for selecting and expelling a dosage with the dial grip. This dial grip may be for example manufactured a single-piece moulded grip and may perform the basic functionality of a dummy dial grip without any special functionality. It may be for example used for shipping injection devices and to provide patients with a basic functionality to select a dose to be injected by turning with the handling part the dial grip when attached to an injection device and expelling the selected dose by pushing the dial grip inwardly. The dial grip may be particularly provided for a releasable attachment to an injection device in order to enable a replacement by another dial grip, which may comprise more functionality.

In embodiments, the dial grip may comprise at least one locking lever being movable between a lock and an unlock position, wherein the at least one locking lever can engage with at least one locking lever reception provided within a body of the injection device when moved in the lock position. The locking lever may for example be provided for locking a turning of the dial grip when the dial grip is positioned on an injection device so that the locking lever is moved in the lock position. After positioning the dial grip to move the locking lever in an unlock position, it may be turned to allow a dose selection. The term locking lever as used herein also comprises a locking bolt, a locking pin, or a locking arm.

In embodiments, the handling part may be configured for a releasable attachment of a grip top. Thus, the handling part allows to attach and detach a grip top from an injection device and, thus, to extend the functionality of the dial grip for example with dose recording when the grip top contains electronics provided and configured for this purpose. When at least one locking lever is comprised by the dial grip, the dial grip may be configured to move the at least one locking lever between the lock and unlock position depending on whether a grid top is attached to the handling part or not. For example, an injection device with an attached dial grip may be shipped without an attached grip top so that the at least one locking lever is moved in the lock position and may prevent that the dial grip can be turned to select a dose. When a grip top is attached to the dial grip, the at least one locking lever may be moved in the unlock position allowing to turn the dial grip and, thus, enabling a patient to select and expel a dosage with the injection device.

In further embodiments, the handling part of the dial grip may comprise fixture for the releasable attachment of the grip top. The grip top may be provided for further enhancing usage of the dial grip and may in certain embodiments be required to allow a dose selection by turning the dial grip. Particularly, a grip top may provide further functionality to the dial grip. A grip top may for example comprise electronics to provide further functionality, may have a special shape or color to improve usage of the dial grip, and/or may comprise additional features useful for patients. In yet further embodiments, the fixture may comprise at least one guide groove and the grip top may comprise one or more guide pins provided on its inside for engaging with the at least one guide groove. A guide groove and guide pin allow to safely fix the grip top to the handling part with moderate technical efforts. Particularly, a guide groove and guide pin may be molded with the handling part and grip top, respectively.

In still further embodiments, the at least one guide groove may comprise an end section and the fixture may further comprise holder being arranged in the end section, wherein the guide pin can be seated in the end section and releasably fixed with the holder in the end section. This allows to more securely fixing the grip top to the handling part. In still further embodiments, the fixture may further comprise a retention spring being arranged in the handling part to exert a force on the holder. Thus, for detaching the dial top from the handling part, the exerted force of the retention spring has to be overcome so that the force required for detaching the grip top may be setup by means of the retention spring and its exerted force.

In embodiments with the dial grip comprising the at least one locking lever being movable between a lock and an unlock position, the retention spring may be arranged to force the at least one locking lever in the lock position when no guide pin is seated in the end section. Thus, the usage of the dial grip, particularly its turning for dose selecting, may be blocked when no grip top is attached to the handling part, or in other words, only with an attached grid top a dose may be selected by turning the dial grip.

In embodiments, the grip base may further comprise a collar coaxially arranged around the clutch and being provided for insertion between a dial sleeve of the dosage selection and expelling mechanism and a body of the injection device. The collar may serve to align the dial grip with the injection device upon attachment to it. Yet further, the collar may also increase stability of the coupling of the dial grip to the injection device in addition to the clutch. In embodiments with the dial grip comprising the at least one locking lever being movable between a lock and an unlock position, the collar may be configured as guiding for the at least one locking lever. This may be particularly useful when the at least one locking lever comprises an elongation, which could make it difficult to attach the dial grip to the injection device. In such a case, the collar used as guiding for the at least one locking lever may make it easier for example for patients to attach the dial grip to the injection device. Particularly, the at least one locking lever may comprise at least one blocking lever and the collar may comprise a groove or channel provided in the collar as guiding for the at least one locking lever.

In a further aspect the present disclosure provides an exchangeable grip top for a dial grip as disclosed herein and comprising at least one guide pin provided on its inside for engaging with at least one guide groove comprised by an handling part of the dial grip. The grip top may be for example manufactured by molding, and the at least one guide pin may be molded on its inside. The grip top may be shaped for a comfortable usage by patients, for example comprise one or more handles for making turning and dose selecting easier for users than with a dial grip without the grid top.

In embodiments, the grip top as disclosed herein or the dial grip as disclosed herein may further comprise electronics configured for at least one of recording, storing, processing, transmitting one or more drug dosages selected and expelled with an injection device. The electronics may be for example placed in the interior of the grid top, for example in a dedicated space under the grip top's end section such that when the grip top is attached to the handling part the electronics is accommodated in a space between the end section of the grip top and the top side of the handling part. Other accommodations of the electronics within the grip top are also possible.

The electronics may be also placed directly in the handling part, for example in a space inside the handling part provided for the electronics.

In further embodiments of the grip top as disclosed herein or the dial grip as disclosed herein, the electronics may be configured to provide one or more of the following functions: Bluetooth® connectivity; Near Field Communication connectivity; WiFi™ connectivity. Thus, the electronics may implement one or more connectivity features to the dial grip allowing transmitting data to and/or receiving data from an external device such as a computer.

In still further embodiments, the grip top as disclosed herein or the dial grip as disclosed herein may comprise a battery as power supply for the electronics, for example a button cell. The battery may particularly be a rechargeable battery and contacts may be provided on the outside of the grip top and/or the dial grip for charging the battery. For example, the grip top or the dial grip may be put into a charging station, in which charging contacts are arranged, which contact the contacts of the grip top or dial grip for supplying the rechargeable battery with a charging current. In particularly embodiments, the electronics may be configured for data communication over the charging contacts. Thus, the charging station may be used as a kind of docking station for charging and data exchange between the electronics of the grip top or dial grip and an external device communicatively coupled with the charging or docking station.

In a yet further aspect the present disclosure provides an injection device comprising a body housing a dosage selection and expelling mechanism having a mechanical interface on one open end of the body, and a dial grip as disclosed herein, wherein a clutch of the dial grip are coupled to a dose selecting part of the dosage selection and expelling mechanism forming the mechanical interface so to allow for dosage selection and expelling with the dial grip. The dial grip may be for example a dummy dial grip provided for shipping without further functionality, and injections devices with such a dummy dial grip may be provided for distribution purposes. The dial grip may be particularly releasably attached to the body of the injection device in order to allow a replacement with another dial grip with different, particularly additional functionality.

In further embodiments, the injection device may comprise a dial grip with the further features as disclosed herein and/or a grip top with the further features as disclosed herein.

According to an embodiment, the present disclosure relates to an injection device comprising a body provided for housing a dosage selection and expelling mechanism, and a dial grip, wherein the dial grip is coupled to the dosage selection and expelling mechanism forming a mechanical interface so to allow for dosage selection with the dial grip, wherein the dial grip is releasable from the dosage selection and expelling mechanism such that the dial grip can be replaced by a second dial grip or a grip top of the dial grip is releasable from the dial grip such that it can be replaced by a second grip top. The second dial grip/replacement dial grip or the second grip top/replacement grip top may provide additional features to the user of the injection device such as a connectivity feature.

The second dial grip or the second grip top may comprise electronics configured for at least one of measuring, recording, storing, processing, or transmitting data related to one or more drug dosages selected or expelled with the injection device.

The electronics may be configured to provide one or more of the following functions: Bluetooth® connectivity; Near Field Communication connectivity; WiFi™ connectivity.

The second dial grip or the second grip top may further comprise a battery as power supply for the electronics, particularly a rechargeable battery and may comprise contacts provided on the outside for charging the battery, wherein the electronics may be configured for data communication over the charging contacts.

The dial grip of the injection device according to this embodiment may be combined with further features disclosed in the description. It may or may not have a grip base. It may or may not have fixture for releasable attachment of a grip top.

### BRIEF DESCRIPTION OF THE FIGURES

FIG. 1 shows an example of an injection pen with a dummy dial grip;

FIG. 2 shows an example of an exchangeable dial grip for an injection device in different views;

FIG. 3 shows an example of dial grip attached to an injection pen in different views; and

FIG. 4 shows the guiding of a locking lever in an example of a dial grip in different views.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen.

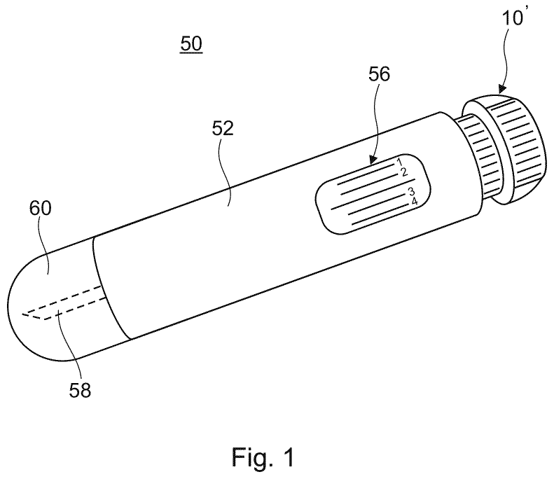

FIG. 1 shows an injection device in the form of an injection pen 50 having body 52. The body 52 is provided for holding a drug cartridge (not shown) and housing a dose selecting and expelling mechanism 56 (from which only a part 56 for dose selection is shown in FIG. 1). The body 52 has a proximal end and a distal end. A syringe 58 protected by a cap 60 is provided at the proximal end of the body 51, and a dummy dial grip 10′ is attached to the distal end of the body 52. The dummy dial grip 10′ may be releasably attached to the distal end, and may provide a basic functionality for a patient as will be described hereinafter.

A patient may use the dummy dial grip 10′ to select a dose. The selected dose is shown on the scale of the dose selecting and expelling mechanism 56. The selection of a dose may be only possible, when a grip top is attached to the dummy dial grip 10′. Locking levers may prevent dose selection when the dummy dial grip 10′ is in a certain attachment position, and/or when no grid top is attached to the dummy dial grip 10′. Dose selection may be typically performed by turning the dummy dial grip 10′ with the attached grip top around its longitudinal axis relative to the body 52. Turning the dummy dial grip 10′ may cause the dose selecting and expelling mechanism to bring a piston in a position, in which the selected dose may be expelled from the cartridge within the body 52. The selected dose may be expelled by pressing the dummy dial grip 10′ down, i.e., in an axial direction to the distal end of the body 52. The force put on the dummy dial grip 10′ by pressing it down may be converted by the dose selecting and expelling mechanism into a pressure pulse being effective on the cartridge, for example on a diaphragm of the cartridge, so that the inner pressure of the cartridge is increased and an amount of the drug contained in the cartridge corresponding to the selected dose is expelled via the syringe 58 into the patient's body.

The dummy dial grip 10′ may be used for shipping the injection pen 50, and a simple grip top may be provided or enclosed enabling dose selecting and expelling as described above. The simple grip top may be part of the dummy dial grip 10′, or it may be a separate part. If the simple grip top is embodied as separate part, it may be replaceable with another grip top to provide further functionality to the injection pen 50. The dummy dial grip 10′ and simple grip top may be implemented with relatively simple technical means, for example as a one-piece molded part, which may be enclosed with a shipment of the injection pen 50 to allow patients to immediately use the pen 50. When patients desire more functionality, the dummy dial grip 10′ and/or the simple grip top may be replaced with a more complex dial grip or grip top, respectively. In order to accomplish this, the dummy dial grip 10′ may be provided for releasable attachment to the injection pen 50 and/or for releasable attachment of a grid top allowing a simple replacement of grip tops.

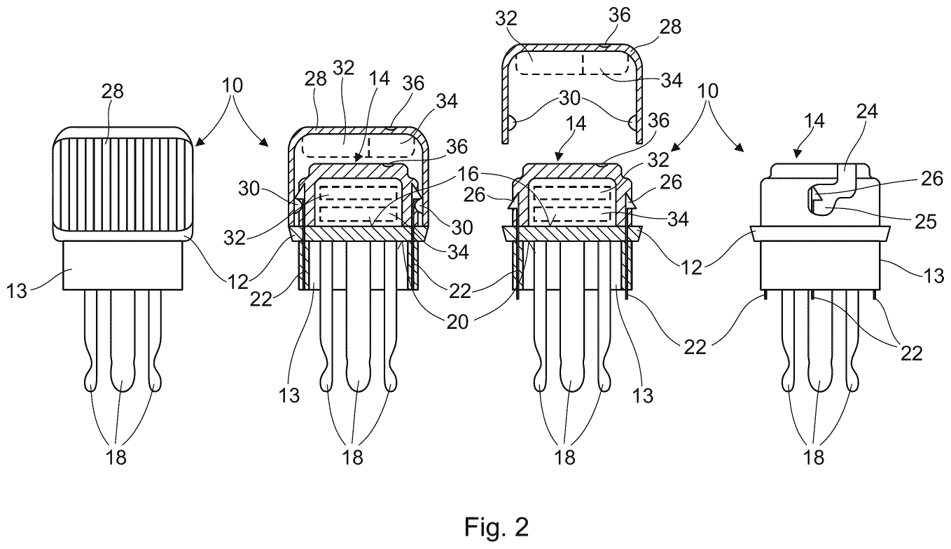

FIG. 2 shows another dial grip 10 for an injection pen in four different views from left to right: a side view of the dial grip 10 with an attached grip top 28; a cross section through the dial grip 10 with the attached grip top 28 showing internals of the attachment of the grip top 28 to the dial grip 10 and of locking levers 22; a cross section through the dial grip 10 with the detached grip top 28 showing internals of the grip top 28 and the locking levers 22; and a side view of the dial grip 10 without the grid top 28 showing details of a guide groove 24 provided in an handling part 14 of the dial grip 10. This dial grip 10 is provided for exchangeable grip tops 28 and can offer additional functionality to patients as will be described below. It may be for example offered as a replacement of a dummy dial grip if patients wish for example a more comfortable handling of an injection pen. However, in principle this dial grip 10 may also be shipped with an injection pen as a kind of dummy dial grip and being extendable with attachable and replaceable grip tops.

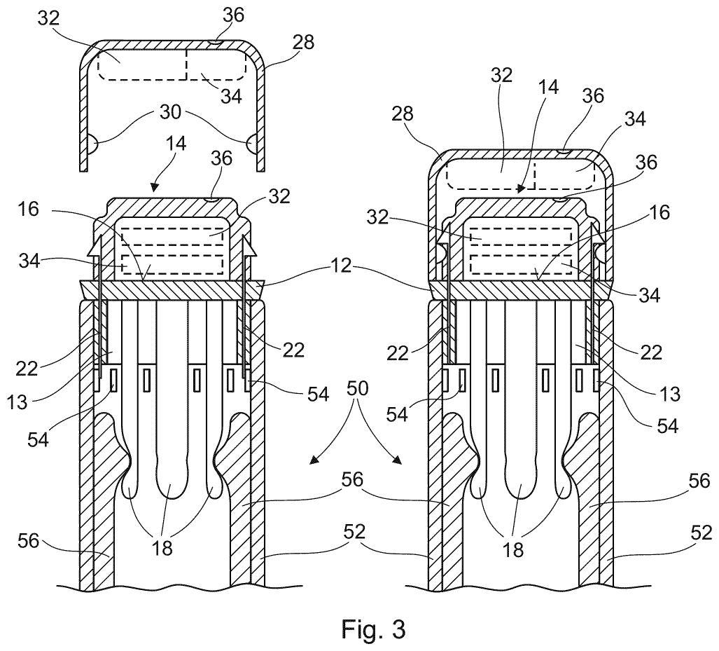

The most right drawing in FIG. 2 shows the dial grip 10 in a side view: a grip base 12 form the middle of the grip 10. The grip base 12 may be shaped like a coin with a diameter similar to the diameter of a distal end of an injection pen, at which the dial grip 10 may be attached. The grip base 12 comprises a handling side 16 and a lower side 20, both arranged opposite to each other like both sides of a coin. The lower side 20 is provided for abutting with a distal end of an injection pen's body 52 (as shown in FIG. 3), and the handling side 16 is provided for attaching a grip top 28 to the handling part 14 protruding from the handling side 16.

A handling part 14 is provided on the handling side 16. The handling part 14 may be formed like a cap extending from the handling side 16 of the grip base 12. The handling part 14 may be hollow to accommodate for example electronics. One or more guide grooves 24 may be provided in the handling part 14, particularly in its sidewall as shown in the most right drawing of FIG. 2. The guide groove 24 may be J-shaped with an open entry on one end of the J and an end section 25 extending from the other end of the J. The end section 25 may serve as a bed for a guide pin 30 of a grip top 28. Holders 26 are arranged in the end section 25 to releasably fix a guide pin 30 in the end sections.

A clutch 18 particularly comprising one or more clamp feet extends from the lower side 20 of the grip base 12. The clutch 18 is configured to engage with a dose selecting part of a dosage selection and expelling mechanism of the injection pen such that a turning of the dial grip 10 around its axis corresponding to the pen's axis incurs a rotation in the dosage selection and expelling mechanism of the injection pen for selecting a desired dose to be expelled. A collar 13 is arranged coaxially around the clutch 18. The collar 13 may serve as an aligner for the dial grip 10 during attachment to the injection pen and when being attached, as will be described later. Furthermore, the collar 13 may serve as a guiding for one or more locking levers 22, as will also be described later in detail.

The most left drawing in FIG. 2 shows the dial grip 10 with attached grip top 28 in a side view. The grip top 28 may comprise a grooved or ribbed surface making it easier to turn the grip top 28. The grip top 28 can be releasably attached to the dial grip 10 as the two cross-sectional side views in FIG. 2 show.

The grip top 28 comprises guide pins 30 arranged at its interior, as can be seen in the middle right drawing. The guide pins 30 may engage with the guide groves 24 when the grip top 28 is put over the handling part 14. The guide pins 30 are arranged to match with the guide grooves 24 such that when the grip top 28 is put over the handling part 14, the guide pins 30 may enter the entries of the guide grooves 24 and may be moved in the grooves 26 to the end section 25 by turning the grip top 28 on the handling part 14 around its axis in a clockwise direction (or counter clockwise direction depending on the arrangement of the guide grooves 24). The guide pin 30 then stops at the left bottom of the J-shaped guide groove 24, i.e., the grip top 28 cannot be turned further. By slightly pushing the grip top 28 in its axial direction on the dial grip 10 to the grip base 12, the guide pin 30 may be forced into the end section 25. In the end section 25, the guide pin 30 may be locked with the holder 26. The holder 26 may be hook-like shaped so that a smaller force may be required for putting the guide pins 30 into the end sections 25 than the force required to pull the guide pins 30 out of the end sections 25.

The holder 26 may be part of the locking lever 22, which may be embodied as levers with the holder 26 forming one end and the other end sticking out of the pen-side-end of the collar 13 when no grip top 28 is attached to the handling part 14 (as shown in the two right drawings of FIG. 2). Each locking lever 22 is guided by the collar 22 and movable between a lock position (shown in the two right drawings of FIG. 2) and an unlock position (shown in the left drawings of FIG. 2). Each locking lever 22 is moved in the unlock position when the grip top 28 is fully attached to the handling part 14, which means that guide pins 20 of the grip top 28 are latched in the end sections 25 of the guide grooves 24 and hold by the holder 26. In such a state, the guide pins 30 lift the holder 26 a little bit which causes the movement of the locking lever 22 from its lock into its unlock position.

The two middle drawings of FIG. 2 also show that electronics 32 and a particularly rechargeable battery 34 may be comprised by the grip top 28 and/or the handling part 14. The electronics 32 may be configured for at least one of recording, storing, processing, or transmitting one or more drug dosages selected and expelled with an injection device 50. Also, contacts 36 may be provided at the outside of the grip top 28 and/or the handling part 14. The contacts 36 may be provided for charging the battery 34, but may also be provided for data exchange of the electronics 32 with external devices. For example, the contacts 36 may be electrically connected to counter contacts of a charging station (not shown), which may be connected to a computer via a data cable such as an USB (Universal Serial Bus) connection, which may be used to supply power for charging and exchange data between the computer and the electronics 32. Thus, the grip top 28 or the dial grip 10 may be connected with an external device for data exchange. The electronics 32 may however also be configured for a wireless data with external devices, for example by implementing Bluetooth® connectivity, Near Field Communication (NFC) connectivity, and/or WiFi™ connectivity, thus allowing to wirelessly exchange data with external devices such as smartphones or tablet PCs. The contacts 36 are optional and may be only provided when a rechargeable battery 34 is applied. Instead of a rechargeable battery, a disposable battery such as a button cell may be used.

FIG. 3 shows the dial grip 10 attached to the distal end of the injection pen body 52. The grip base 12 abuts the end of the sleeve of the body 52. The collar 13 aligns the attached dial grip 10 in the body sleeve 52. The clutch 18 of the dial grip 10 engages with a dose selecting part 56 of the dose selection and expelling mechanism such that the clutch 18 and dose selecting part 56 are rotationally coupled.

In the left drawing of FIG. 3 the grip top 28 is not attached to the dial grip 10 so that the locking lever 22 is in the lock position, i.e., one end sticks out of the collar's 13 pen-sided end. The stick out end of the locking lever 22 lever engages with a locking lever reception 54 arranged on the inside of the pen's body sleeve 52. The locking lever reception 54 may be for example embodied as a crown blocking a rotation of the dial grip 10 relative to the pen's body sleeve 52 so that also the dose selecting part 56 cannot be turned and no dose selection is possible. Other implementations of the locking lever 22 and the locking lever reception 54 are possible as long as the function of blocking a rotation of the dial grip 10 relative to the pen's body sleeve 52 is fulfilled when no grip top 28 is attached to the dial grip 10.

The right drawing of FIG. 3 shows the grip top 28 fully attached to and locked with the dial grip 10. In this state, the guide pins 30 are seated in the end sections 25 of the guide grooves 24 (FIG. 2) and lift the holder 26 at one end of the locking lever 22 lever so that the locking lever 22 are brought into the unlock position, in which they no longer engage with the locking lever reception 54 on the inside of the pen's body sleeve 52. Thus, a rotation of the dial grip 10 with the attached grip top 28 relative to the pen's body sleeve 52 is possible, and, therefore, a dose can be selected by rotating the dose selecting part 56 rotationally coupled to the clutch 18 together with the rotation of the dial grip 10.

As can be seen in FIG. 3, the clutch 18 and the dose selecting part 56 may be designed for clicking in each other when the dial grip 10 is attached to the body 54, i.e., when the collar 13 is inserted into the body sleeve 54 until the grip base 12 abuts the sleeve's end. This allows removing the dial grip 10 from the pen's body 54 by pulling it out of the body sleeve 54 with a force large enough to overcome the click forces of both the clutch 18 and the dose selecting part 56.

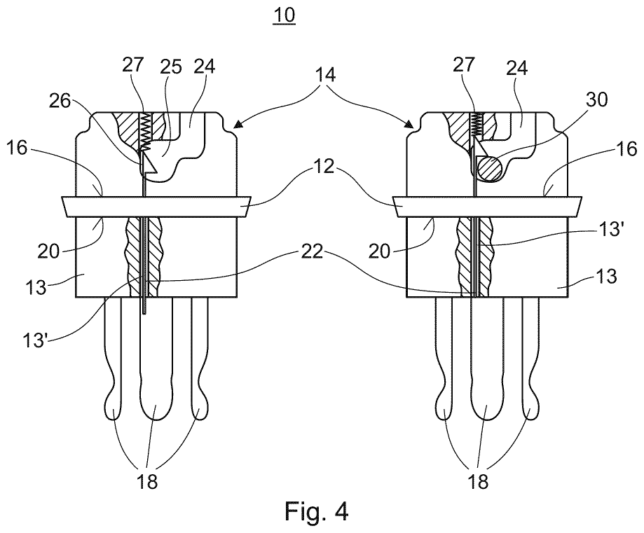

FIG. 4 shows in a partly cross-section the guiding of the locking lever 22 by the collar 13: the lever part of the locking lever 22 is movably placed in a channel 13′ of the collar. Instead of a channel 13′, a groove may be also used as guiding for the lever part of the locking lever 22. The hook-like shaped holder 26 at the handling end of the locking lever 22 rests in the end section 25 of the guide groove 24. A retention spring 27 is placed above the holder 26 in a seat within the handling part 14 and puts a force on the holder 26 pressing the locking lever 22 down so that the lower end of the locking lever 22 picks out of the collar 13 and the channel 13′ for engagement with a locking lever reception 54 inside the body 52 of the injection pen (see FIG. 3).

It should be noted that even if the retention spring 27 is shown in an angular orientation, it could be also arranged in another orientation, for example a radial orientation pushing the holder outward.

It should be also noted that also another arrangement of the locking lever 22 is possible, for example hinged levers, as long as an arrest between the pen's body 52 and the dial grip 10 is obtained when the grip top 28 is removed.

The right drawing of FIG. 4 shows a state with a guide pin 30 resting in the end section 25 of the guide groove 24 when the grip top 28 is fully attached to the dial grip 10 (as shown in the right drawing in FIG. 3). The guide pin 30 is hold by the holder 26 in its position in the end section 25, and the holder 25 is upwardly pressed by the guide pin 30 against the force of the retention spring 27. This also causes an upward movement of the locking lever 22 so that it is entirely rested in the channel 13′ and no part of the locking lever 22 picks out of the collar 13 at its bottom. In this state, the dial grip 10 together with the fully attached grip top 28 can be turned around its longitudinal axis relative to the body 52 of the injection pen, and the moving clutch 18 can move the rotationally coupled dose selecting part 56 for selecting a dose.

The grip top 28 can be detached from the dial grip 10 be pulling it in an axial direction a little bit away from the grip base 12 so that the guide pin 30 is moved out of the end section 25, which requires a force larger than the force of the retention spring 27 is putting on the holder 26. As soon as the guide pin 30 is moved out of the end section 25, the retention spring 27 can expand and force the holder 26 and the locking lever 22 downward (as shown in the left drawing in FIG. 4) so that a relative movement of the dial grip 10 with regard to the body 52 of the injection pen is blocked and the further handling of the grip top 28 cannot result in an unintentional dose selecting or expelling. When the guide pin 30 can then be moved out of the end section 25, and the grip top 28 can be turned counter-clockwise to move the guide 30 in the section of the guide groove 24, which is essentially parallel to the grip base 12, until the guide pin 30 adjoins the axial section of the guide groove 24, which is open at the top of the handling part 14. The grip top 28 can finally be detached by pulling it axially away from the handling part 14 of the dial grip 10.

A detachment of the dial grip 10 from the injection pen's body 52 can be performed by turning the dial grip 10 to a 0IU stop of the dose selecting and expelling mechanism of the injection pen, then pulling the dial grip 10 axially outward and twisting the dial grip 10 further (over the 0IU stop, with the pen's mechanism staying at 0IU). Lastly, the dial grip 10 can be removed from the body 52 by pulling it axially off. This may be performed for replacing for example a dummy dial grip with a dial grip comprising additional functionality such as electronics.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia. Examples of DPP4 inhibitors are Vildagliptin, Sitagliptin, Denagliptin, Saxagliptin, Berberine. Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1-16. (canceled)

17. A dial grip for an injection device, the dial grip comprising:
a grip base,
a handling part provided on one side of the grip base, and
a clutch being provided on the other side of the grip base for engagement with a dose selecting part of a dosage selection and expelling mechanism of the injection device to couple the grip base to the dosage selection and expelling mechanism for selecting a dosage with the dial grip.

18. The dial grip of claim 17, comprising at least one locking lever being movable between a lock position and an unlock position, wherein the at least one locking lever can engage with a locking lever reception provided within a body of the injection device when moved in the lock position.

19. The dial grip of claim 17, wherein the handling part is configured for a releasable attachment of a grip top.

20. The dial grip of claim 19, wherein the handling part comprises at least one fixture for the releasable attachment of the grip top.

21. The dial grip of claim 20, wherein the at least one fixture comprises at least one guide groove and the grip top comprises one or more guide pins provided on its inside surface for engaging with the at least one guide groove.

22. The dial grip of claim 21, wherein the at least one guide groove comprises an end section and the at least one fixture further comprises a holder being arranged in the end section, wherein the one or more guide pins can be seated in the end section and releasably fixed with the holder in the end section.

23. The dial grip of claim 22, wherein the at least one fixture further comprises a retention spring being arranged in the handling part to exert a force on the holder, the retention spring being arranged to force at least one locking lever of the dial grip into a lock position when no guide pin is seated in the end section.

24. The dial grip of claim 17, wherein the grip base further comprises a collar coaxially arranged around the clutch and being provided for insertion between a dial sleeve of the dosage selection and expelling mechanism and a body of the injection device, the collar being configured as a guide for at least one locking lever of the dial grip.

25. The dial grip of claim 17, further comprising electronics configured for at least one of recording, storing, processing, or transmitting one or more drug dosages selected and expelled with an injection device.

26. The dial grip of claim 25, wherein the electronics are configured to provide one or more of the following functions: Bluetooth® connectivity; Near Field Communication connectivity; or WiFi™ connectivity.

27. The dial grip of claim 25, further comprising:
a battery as power supply for the electronics, the battery including a rechargeable battery, and
contacts provided on an outside surface of the dial grip for charging the battery,
wherein the electronics are configured for data communication over the contacts.

28. An exchangeable grip top for a dial grip, the dial grip comprising:
a grip base,
a handling part provided on one side of the grip base, and
a clutch being provided on the other side of the grip base for engagement with a dose selecting part of a dosage selection and expelling mechanism of an injection device to couple the grip base to the dosage selection and expelling mechanism for selecting a dosage with the dial grip,
the grip top comprising: at least one guide pin provided on an inside surface of the grip top for engaging with at least one guide groove included in the handling part of the dial grip.

29. The grip top of claim 28, further comprising electronics configured for at least one of recording, storing, processing, or transmitting one or more drug dosages selected and expelled with an injection device.

30. The grip top of claim 29, wherein the electronics are configured to provide one or more of the following functions: Bluetooth® connectivity; Near Field Communication connectivity; or WiFi™ connectivity.

31. The grip top of claim 29, further comprising:
a battery as power supply for the electronics, the battery including a rechargeable battery, and
contacts provided on an outside surface of the grip top for charging the battery,
wherein the electronics are particularly configured for data communication over the contacts.

32. An injection device comprising:
a body provided for housing a dosage selection and expelling mechanism, and
a dial grip, wherein the dial grip is coupled to the dosage selection and expelling mechanism forming a mechanical interface to allow for dosage selection with the dial grip,
wherein the dial grip is releasable from the dosage selection and expelling mechanism such that the dial grip can be replaced by a second dial grip, or
a grip top of the dial grip is releasable from the dial grip such that it can be replaced by a second grip top.

33. The injection device of claim 32, wherein the second dial grip or the second grip top comprises electronics configured for at least one of measuring, recording, storing, processing, or transmitting data related to one or more drug dosages selected or expelled with the injection device.

34. The injection device of claim 32, wherein the dosage selection and expelling mechanism having a mechanical interface on one open end of the body, and
wherein a clutch of the dial grip is coupled to a dose selecting part of the dosage selection and expelling mechanism forming the mechanical interface to allow for dosage selection and expelling with the dial grip.

35. The injection device of claim 34, wherein the dial grip comprises:
a grip base,
a handling part provided on one side of the grip base, and
the clutch being provided on the other side of the grip base to couple the grip base to the dosage selection and expelling mechanism for selecting a dosage with the dial grip.

36. The injection device of claim 35, wherein the grip top comprises at least one guide pin provided on an inside surface of the grip top for engaging with at least one guide groove included in the handling part of the dial grip.
